(19) United States
(12) Patent Application Publication
(10) Pub. No.: US 2025/0352580 A1
(43) Pub. Date: Nov. 20, 2025

(54) COMPOSITIONS AND METHODS FOR B CELL DIRECTED IMMUNOTHERAPIES FOR ANTI-AAV NEUTRALIZING ALLOANTIBODIES

(71) Applicants: The Children's Hospital of Philadelphia, Philadelphia, PA (US); The Trustees of the University of Pennsylvania, Philadelphia, PA (US)

(72) Inventors: Bhavya Doshi, Philadelphia, PA (US); Lindsey A. George, Haddonfield, NJ (US); Joseph Fraietta, Philadelphia, PA (US); Vijay Bhoj, Philadelphia, PA (US)

(21) Appl. No.: 19/120,347

(22) PCT Filed: Nov. 6, 2023
(86) PCT No.: PCT/US23/78799

§ 371 Date: Apr. 11, 2025

Related U.S. Application Data
(60) Provisional application No. 63/422,596, filed on Nov. 4, 2022.

(51) Int. Cl. A61K 35/17; A61K 40/11; A61K 40/42; A61P 37/06; C07K 16/28; C12N 15/86

(52) U.S. Cl. CPC A61K 35/17; A61K 40/11; A61K 40/4211; A61P 37/06; C07K 16/2803; C07K 16/2878; C12N 15/86; C12N 2750/14143

(57) ABSTRACT

Compositions and methods for depleting B cells and/or eliminating neutralizing antibodies, particularly for AA V gene therapy administration, are disclosed. Neutralizing alloantibodies (NAbs) to adeno-associated virus (AAV) occur both naturally after exposure to the wildtype circulating virus and following AAV vector mediated gene therapies. When pre-existing, these NAbs preclude eligibility to receive curative AAV gene therapy vectors. Following AAV infusion, the development of NAbs preclude re-administration even with other vector serotypes due to cross-reactivity. Given waning of transgene activity seen in multiple AAV gene therapy trials for hemophilia and the potential need to boost responses with growth in pediatric patients, the ability to re-administer AAV vectors is critical.

This application claims priority under 35 U.S.C. § 119 (e) to U.S. Provisional Patent Application No. 63/422,596, filed Nov. 4, 2022. The foregoing application is incorporated by reference herein.

### FIELD OF THE INVENTION

The present invention relates to the fields of medicine and immunology. More specifically, the invention provides compositions and methods for B cell directed immunotherapies for neutralizing alloantibodies.

### BACKGROUND OF THE INVENTION

Several publications and patent documents are cited throughout the specification in order to describe the state of the art to which this invention pertains. Each of these citations is incorporated herein by reference as though set forth in full.

Neutralizing alloantibodies (NAbs) to adeno-associated virus (AAV) occur both naturally after exposure to the wildtype circulating virus and following AAV vector mediated gene therapies. When pre-existing, these NAbs preclude eligibility to receive curative AAV gene therapy vectors. Following AAV infusion, the development of NAbs preclude re-administration even with other vector serotypes due to cross-reactivity. Given waning of transgene activity seen in multiple AAV gene therapy trials for hemophilia and the potential need to boost responses with growth in pediatric patients, the ability to re-administer AAV vectors is critical. Limited pre-clinical evaluations have assessed anti-CD20 (mature B cell antibody) (Mingozzi, et al. (2013) Gene Ther., 20:417-424), plasmapheresis (Bertin, et al. (2020) Sci. Rep., 10:864; Chicoine, et al. (2014) Mol. Ther., 22:338-47; Monteilhet, et al. (2011) Mol. Ther., 19:2084-91), rapamycin (Meliani et al. (2018) Nat. Commun., 9:4098), and an IgG cleaving endopeptidase (Leborgne, et al. (2020) Nature Medicine 26:1096-1101; Elmore, et al. (2020) JCI Insight 5 (19): e139881) for NAb eradication, but these methods have had limited success. Therefore, there is a clear need for methods for eliminating anti-AAV NAbs.

### SUMMARY OF THE INVENTION

In accordance with the present invention, compositions and methods for reducing neutralizing antibodies and/or depleting B cells are provided. In certain embodiments, the method comprises administering a CD19 targeting agent, a B-cell activating factor (BAFF) targeting agent, a B-cell maturation antigen (BCMA) targeting agent, and/or a transmembrane activator and CAML interactor (TACI) targeting agent to a subject, cell, or tissue. In certain embodiments, the targeting agent results or effects the death of B cells. In certain embodiments, the method comprises administering a CD19 targeting agent. In certain embodiments, the method comprises administering a CD19 targeting agent and a BCMA targeting agent. In certain embodiments, the neutralizing antibodies are anti-adeno-associated virus (AAV) neutralizing antibodies. In certain embodiments, the titer of neutralizing antibodies is reduced to <1:10. In certain embodiments, the B cells are plasma cells. In certain embodiments, the targeting agents are selected from the group consisting of antibodies, antigen binding antibody fragments, immunotoxins, antibody drug conjugates, small molecules, cytotoxic agents, and chimeric antigen receptor T cells (CAR-T). In certain embodiments, the targeting agents comprise monoclonal antibodies. In certain embodiments, the targeting agents induce antibody-dependent cellular cytotoxicity (ADCC) and/or complement-dependent cytotoxicity (CDC).

In accordance with another aspect of the instant invention, compositions and methods are provided for administering a viral vector to a subject. In certain embodiments, the method comprises i) administering a CD19 targeting agent, a BAFF targeting agent, a BCMA targeting agent, and/or a TACI targeting agent to the subject; and ii) administering the viral vector to the subject. In certain embodiments, the viral vector is an AAV vector (e.g., AAV gene therapy vector). The viral vector administration may be an initial therapy or a re-administration of the therapy. In certain embodiments, step i) occurs before step ii). In certain embodiments, the subject has neutralizing antibodies (e.g., anti-AAV neutralizing antibodies) against the viral vector prior to treatment. In certain embodiments, the method comprises administering a CD19 targeting agent. In certain embodiments, the method comprises administering a CD19 targeting agent and a BCMA targeting agent. In certain embodiments, the targeting agents are selected from the group consisting of antibodies, antigen binding antibody fragments, immunotoxins, antibody drug conjugates, small molecules, cytotoxic agents, and CAR-T. In certain embodiments, the targeting agents comprise monoclonal antibodies. In certain embodiments, the targeting agents induce antibody-dependent cellular cytotoxicity (ADCC) and/or complement-dependent cytotoxicity (CDC).

### BRIEF DESCRIPTION OF THE DRAWINGS

FIG. 1 provides a graph of AAV8 NAbs over time in mice administered non-transduced controls (NTD) CAR-T (control), BCMA CAR-T, or CD19 CAR-T and BCMA CAR-T.

FIG. 2 provides a graph of AAV8 NAbs over time in mice administered NTD CAR-T, BCMA CAR-T, CD19 CAR-T, or CD19 CAR-T and BCMA CAR-T.

FIG. 3 provides a graph of the amount of B cells in mice 4 weeks after treatment with NTD CAR-T, BCMA CAR-T, CD19 CAR-T, or CD19 CAR-T and BCMA CAR-T.

FIGS. 4A-4C show that anti-CD19 containing therapy effectively depletes AAV NAb. FIG. 4A provides a schematic of AAV immunization and CAR-T therapy. C57B1/6 mice were immunized with AAV8 at 1×10¹¹ vg/mouse two weeks prior to total body irradiation followed by CAR-T infusion. Four CAR constructs were used including NTD (controls), BCMA, CD19, and CD19+BCMA. Peripheral blood was collected every two weeks for CD19+ B cell quantitation by flow cytometry (FIG. 4B) and AAV NAb titers by a transduction inhibition assay (FIG. 4C). *** p<0.001.

FIGS. 5A-5D show that anti-CD19 containing CAR-T effectively depletes AAV NAb to permit new AAV transduction. FIG. 5A provides a schematic of AAV re-challenge. Eleven weeks following NTD or CD19 CAR-T therapy, mice were infused with an AAV8 vector carrying a human FVIII transgene at 1×10¹¹ or 1×10¹² vg/mouse. In parallel, control mice that were not previously immunized with AAV were irradiated and infused with CD19 CAR-T followed 11 weeks later by AAV8-hFVIII at the same two doses. FIG. 5B provides a graph of FVIII antigen expression over time following AAV vector rechallenge. hFVIII antigen expression is detectable by week 8 in all mice and approaches steady state by week 10. FIG. 5C provides a graph of FVIII antigen expression at week 10 following AAV challenge. Previously AAV immunized mice (open symbols) approach comparable FVIII antigen levels as seen in previously unimmunized mice (closed symbols) with respect to vector dose given. NTD mice show no hFVIII expression. * p<0.05. FIG. 5D provides a graph of AAV neutralizing antibody titer following AAV vector re-challenge in NTD mice versus CD19 treated mice, n=10-15 mice/cohort. T-test: p<0.05.

FIGS. 6A-6D show B cell and plasma cell quantification following CAR-T therapy. At end of study, animals were euthanized lymphocytes isolated for flow cytometry. Bone marrow total B cells (FIG. 6A) and plasma cells (FIG. 6B) were normalized to million lymphocytes. There was significant B cell aplasia following CD19 containing CAR-T regimens and a trend towards decreased plasma cells from bone marrow with CD19 CAR-T. Splenic total B cells (FIG. 6C) and plasma cells (FIG. 6D) were significantly depleted with CD19 and CD19+BCMA CAR regimens. * p<0.05, **** p<0.0001.

### DETAILED DESCRIPTION OF THE INVENTION

Herein, chimeric antigen receptor T cells (CAR-T) directed against CD19 (pan-B cell marker) and B-cell maturation antigen (BCMA; also known as TNFRSF17) (long-lived plasma cell marker) were administered to a murine model and it was shown that CD19 depletion alone is sufficient for eradication of AAV NAbs to allow AAV vector re-administration. This is the first demonstration of CART-19 technology against neutralizing antibodies and alloantibody responses.

The seroprevalence of anti-AAV NAbs is variable throughout human populations and is dependent on a number of factors including the capsid serotype, geographic region, and age (Boutin, et al. (2010) Hum. Gene Therapy 21:704-712; Kruzik, et al. (2019) Mol. Ther. Methods Clin. Dev., 14:126-133). Generally, NAb against AAV serotype 2 (AAV 2) are the most common and can be found in 50% of adults. AAV 5, 8, and 9 NAbs are less prevalent, but can still be found in up to 40% of adults depending on the serotype.

Inasmuch as AAV serotypes have a high degree of amino acid sequence and structural homology, NAbs generated against one serotype can cross-neutralize other serotypes. Indeed, the presence of anti-AAV NAbs, particularly at a high titer, precludes initial treatment with AAV and precludes vector re-administration (Manno, et al. (2006) Nat Med., 12:342-347; George, et al. (2020) Mol. Ther., 28:2073-2082).

CD20 is a B cell marker that is expressed on pro-B, pre-B, immature, transitional, and memory cells. In contrast, CD19 is also expressed on short-lived plasma cells and plasmablasts. Further, B-cell maturation antigen (BCMA; also known as tumor necrosis factor receptor superfamily member 17 (TNFRSF17)) is expressed on short-lived plasma cells, plasmablasts, and long-lived plasma cells. As such, the targeting of CD19 and/or BCMA allows for the targeting of plasma cells which do not express CD20.

In accordance with an aspect of the instant invention, methods of reducing neutralizing antibodies (e.g., alloantibodies) are provided. The methods can be performed in vivo or in vitro. In certain embodiments, the method is transient (e.g., results in the reduction of neutralizing antibodies for weeks (e.g., 2-12, 4-10, or 6-8 weeks) or months (e.g., 1-3 months)). In certain embodiments, the neutralizing antibody titer is reduced to less than about <1:100, <1:50, <1:25, <1:10, or <1:5. In certain embodiments, the neutralizing antibodies are anti-AAV neutralizing antibodies. In certain embodiments, the method comprises administering a B-cell depleting agent. In certain embodiments, the methods comprise administering a CD19 targeting agent to a subject. In certain embodiments, the methods comprise administering or delivering a CD19 targeting agent to a cell or tissue (e.g., in vitro). In certain embodiments, the methods further comprise administering a B-cell maturation antigen (BCMA) targeting agent. In certain embodiments, the methods further comprise administering a B-cell activating factor (BAFF; also known as tumor necrosis factor ligand superfamily member 13B and CD257) targeting agent. In certain embodiments, the methods further comprise administering a transmembrane activator and CAML interactor (TACI; also known as tumor necrosis factor receptor superfamily member 13B (TNFRSF13B)) targeting agent. In certain embodiments, the method comprises administering a CD19 targeting agent, a BAFF targeting agent, and a BCMA targeting agent. In certain embodiments, the method comprises administering a CD19 targeting agent, a TACI targeting agent, and a BCMA targeting agent. When more than one targeting agent is being administered, the targeting agents can be administered at the same time (e.g., simultaneously) and/or consecutively. When more than one targeting agent is being administered, the targeting agents can be administered in the same composition or in separate compositions. In certain embodiments, the method further comprises administering at least one additional B-cell depleting agent or therapy. In certain embodiments, the method further comprises measuring the amount or presence of neutralizing antibodies after treatment, optionally at more than one timepoint (e.g., to confirm reduction of neutralizing antibodies).

In accordance with another aspect of the instant invention, methods of depleting or reducing B cells (e.g., including plasma cells) are provided. The methods can be performed in vivo or in vitro. In certain embodiments, B cells are depleted or reduced in lymphoid organs and/or peripheral blood. In certain embodiments, the method is transient (e.g., results in the depletion or reduction of B cells for weeks (e.g., 2-12, 4-10, or 6-8 weeks) or months (e.g., 1-3 months)). In certain embodiments, the B cells produce anti-AAV neutralizing antibodies. In certain embodiments, the methods comprise administering a CD19 targeting agent to a subject. In certain embodiments, the methods comprise administering or delivering a CD19 targeting agent to a cell or tissue (e.g., in vitro). In certain embodiments, the methods further comprise administering a BCMA targeting agent. In certain embodiments, the methods further comprise administering a BAFF targeting agent. In certain embodiments, the methods further comprise administering a TACI targeting agent. In certain embodiments, the method comprises administering a CD19 targeting agent, a BAFF targeting agent, and a BCMA targeting agent. In certain embodiments, the method comprises administering a CD19 targeting agent, a TACI targeting agent, and a BCMA targeting agent. When more than one targeting agent is being administered, the targeting agents can be administered at the same time (e.g., simultaneously) and/or consecutively. When more than one targeting agent is being administered, the targeting agents can be administered in the same composition or in separate compositions. In certain embodiments, the method further comprises administering at least one additional B-cell depleting agent or therapy. In certain embodiments, the method further comprises measuring the amount or presence of B cells (e.g., in lymphoid organs and/or peripheral blood) after treatment, optionally at more than one timepoint (e.g., to confirm depletion or reduction of the B cells).

In accordance with another aspect of the instant invention, methods of administering a viral vector therapy, particularly an AAV based therapy (e.g., administration of a AAV vector) such as an AAV gene therapy vector, to a subject are provided. In certain embodiments, the subject has neutralizing antibodies (e.g., alloantibodies) against the viral vector (e.g., AAV vector).

In certain embodiments, the method comprises i) administering a B-cell depleting agent of the instant invention (e.g. a CD19 targeting agent) to a subject, and ii) administering the viral vector (e.g., AAV based therapy) to the subject. In certain embodiments, step i) occurs at least before step ii). In certain embodiments, step i) occurs at least at the same time as step ii). In certain embodiments, step i) occurs at least 1, 2, 3, 4, 5, 6, 7, 8, 9, 10, 11, 12, 13, 14, 15, 16, 17, 18, 19, 20, 21, 22, 23, 24, 25, 26, 27, 28, or more days before step ii). In certain embodiments, step i) comprises administering a CD19 targeting agent. In certain embodiments, step i) further comprises administering a

BCMA targeting agent. In certain embodiments, step i) further comprises administering a BAFF targeting agent. In certain embodiments, the step i) further comprises administering a TACI targeting agent. In certain embodiments, step i) comprises administering a CD19 targeting agent, a BAFF targeting agent, and a BCMA targeting agent. In certain embodiments, step i) comprises administering a CD19 targeting agent, a TACI targeting agent, and a BCMA targeting agent. In certain embodiments, the method further comprises (e.g., in step i)) administering at least one additional B-cell depleting agent or therapy. In certain embodiments, the method further comprises measuring the amount or presence of B cells (e.g., in lymphoid organs and/or peripheral blood) after step i), optionally at more than one timepoint (e.g., to confirm depletion or reduction of the B cells), optionally before step ii). When more than one targeting agent is being administered, the targeting agents can be administered at the same time (e.g., simultaneously) and/or consecutively. When more than one targeting agent is being administered, the targeting agents can be administered in the same composition or in separate compositions. In certain embodiments, the method further comprises measuring the amount or presence of neutralizing antibodies after step i), optionally at more than one timepoint (e.g., to confirm reduction of neutralizing antibodies), optionally before step ii). In certain embodiments, the method further comprises measuring the amount or presence of neutralizing antibodies prior to step i) (e.g., to determine that the subject has neutralizing antibodies against the viral vector).

The targeting agents of the instant invention may specifically bind the target protein. The targeting agents of the instant invention effect B-cell depletion or reduction. In certain embodiments, the targeting agents are inhibitors of the target protein. Examples of targeting agents include without limitation: antibodies (e.g., monoclonal antibody), antigen binding antibody fragments, immunotoxins (e.g., an antibody or antigen binding fragment thereof conjugated or linked to a cytotoxic agent), antibody drug conjugates, small molecules, cytotoxic agents, and CAR-Ts. In certain embodiments, the targeting agents are CAR-T (e.g., the chimeric antigen receptor specifically binds the target protein). In certain embodiments, the targeting agents are antibodies or antigen-binding fragments thereof which are immunologically specific for the target antibody. In certain embodiments, the antibody or antigen binding fragment thereof (e.g., comprising an Fc domain (e.g., a monoclonal antibody)) induces antibody-dependent cellular cytotoxicity (ADCC) and/or complement-dependent cytotoxicity (CDC). In certain embodiments, the targeting agents are immunotoxins or antibody drug conjugates (ADC). In certain embodiments, the targeting agents are bispecific. For example, the targeting agent may comprise a CD19 targeting agent (e.g., antibody) and a BCMA targeting agent (e.g., antibody), optionally connected via a linker.

Targeting agents which target CD19, BCMA, BAFF, and TACI are known in the art. For example, antibodies (e.g., monoclonal antibodies) against CD19, BCMA, BAFF, and TACI are commercially available (e.g., from ThermoFisher Scientific). Tafasitamab and loncastuximab are examples of anti-CD19 targeted monoclonal antibodies. Belantamab and J22.9-ISY (WO 2018/115466) are examples of anti-BCMA monoclonal antibodies. Belimumab and tabalumab are examples of monoclonal antibodies against BAFF. Loncastuximab tesirine is an antibody drug conjugate (ADC) or immunotoxin comprising an anti-CD19 monoclonal antibody conjugated to a pyrrolobenzodiazepine dimer (PBD) alkylating agent (SG3199) via a protease-cleavable linker. Belantamab mafodotin is an antibody-drug conjugate (ADC) or immunotoxin comprising an afucosylated IgG1 directed against BCMA conjugated to the microtubule inhibitor maleimidocaproyl monomethylauristatin-F (MMAF) via a linker. HDP-101 is an anti-BCMA antibody conjugated to amantin. CC-99712 and MEDI2228 are anti-BCMA antibody-drug conjugates.

Tisagenlecleucel (Kymriah®), lisocabtagene maraleucel (Breyanzi®), axicabtagene ciloleucel (Yescarta®), and brexucabtagene autoleucel (Tecartus®) are examples of CD19 CAR-T therapies (e.g., CAR19 therapy). Idecabtagene vicleucel (Abecma®) and ciltacabtagene autoleucel (Carvykti®) are examples of BCMA CAR-T therapies. Examples of BCMA CAR-T therapies have also been described (see, e.g., Roex et al., Pharmaceutics (2020) 12 (2): 194; Roex et al., J. Hematology Oncology (2020) 13:164; NCT03549442).

As stated hereinabove, the targeting agents of the instant invention may be administered with at least one additional B-cell depleting agent or therapy. Examples of additional B-cell depleting agents and therapies include, without limitation: plasmapheresis, anti-CD20 antibodies (e.g., monoclonal antibodies), rapamycin, and IgG endopeptidases (e.g., imifildase). Examples of anti-CD20 antibodies include, without limitation, rituximab, ocrelizumab, veltuzumab, obinutuzumab, ofatumumab, ibritumomab (e.g., ibritumomab tiuxetan), epcoritamab, ublituximab, zebituzumab, divozilimab, veltuzumab, and glofitamab. When an additional B-cell depleting agent or therapy is being administered, the targeting agent of the instant invention can be administered at the same time (e.g., simultaneously) and/or consecutively with the additional B-cell depleting agent or therapy. When an additional B-cell depleting agent is being administered, the targeting agent of the instant invention can be administered in the same composition or in a separate composition as the additional B-cell depleting agent.

The agents of the instant invention may be administered in a composition further comprising a pharmaceutically acceptable carrier. If multiple agents or compositions are utilized, the agents or compositions may be contained within a kit. Kits comprising at least one targeting agent of the instant invention are encompassed by the instant invention. As used herein, the term “kit” refers to a collection of components for use together. For examples, the kits of the instant invention may be for depleting or reducing B cells. The components of the kit may be packaged together. For example, a kit may comprise a first composition comprising a CD19 targeting agent and a second composition comprising a BCMA or BAFF targeting agent. In certain embodiments, the kit further comprises at least one additional B-cell depleting agent. The components may be contained within separate containers. The components may be lyophilized. The components may be in solution. In certain embodiments, the kit further comprises solutions and/or buffers (e.g., for reconstituting lyophilized components). In certain embodiment, the kits further comprise instruction for using the kit. In certain embodiments, the kit further comprises vials of bottles for mixing of components.

The compositions of the present invention can be administered by any suitable route, for example, by injection (e.g., for local or systemic administration), intravenous, oral, pulmonary, nasal or other modes of administration. In a particular embodiment, the compositions are administered parenterally, subcutaneously, or into the bloodstream. In a particular embodiment, the compositions are administered intravenously. The compositions comprising the agents of the invention may be conveniently formulated for administration with an acceptable medium such as water, buffered saline, ethanol, polyol (for example, glycerol, propylene glycol, liquid polyethylene glycol and the like), dimethyl sulfoxide (DMSO), oils, detergents, suspending agents or suitable mixtures thereof. Selection of a suitable pharmaceutical preparation depends upon the method of administration chosen. The concentration of the agents in the chosen medium may be varied and the medium may be chosen based on the desired route of administration of the pharmaceutical preparation. Except insofar as any conventional media or agent is incompatible with the agents to be administered, its use in the pharmaceutical preparation is contemplated. The pharmaceutical composition of the present invention can be prepared, for example, in liquid form, or can be in dried powder form (e.g., lyophilized).

Pharmaceutical compositions containing agents of the present invention as the active ingredient in intimate admixture with a pharmaceutical carrier can be prepared according to conventional pharmaceutical compounding techniques. The carrier may take a wide variety of forms depending on the form of preparation desired for administration.

A pharmaceutical preparation of the invention may be formulated in dosage unit form for ease of administration and uniformity of dosage. Dosage unit form, as used herein, refers to a physically discrete unit of the pharmaceutical preparation appropriate for the patient undergoing treatment. Each dosage should contain a quantity of active ingredient calculated to produce the desired effect in association with the selected pharmaceutical carrier. Procedures for determining the appropriate dosage unit are well known to those skilled in the art. Dosage units may be proportionately increased or decreased based on the weight of the patient. Appropriate concentrations for alleviation of a particular pathological condition may be determined by dosage concentration curve calculations, as known in the art.

The dose and dosage regimen of the agents according to the invention that is suitable for administration to a particular patient may be determined by a physician considering the patient's age, sex, weight, general medical condition, and the specific condition and severity thereof for which the agent is being administered. The physician may also consider the route of administration of the agent, the pharmaceutical carrier with which the agent may be combined, and the agent's biological activity. The appropriate dosage unit for the administration of the agents of the invention may be determined by evaluating the toxicity of the agents in animal models. Appropriate dosage unit may also be determined by assessing the efficacy of the agents in combination with other standard drugs.

The compositions comprising the agents of the instant invention may be administered at appropriate intervals, for example, at least once a day or more until the pathological symptoms are reduced or alleviated, after which the dosage may be reduced to a maintenance level. The appropriate interval in a particular case would normally depend on the condition of the patient.

### Definitions

The following definitions are provided to facilitate an understanding of the present invention.

The singular forms “a,” “an,” and “the” include plural referents unless the context clearly dictates otherwise.

“Pharmaceutically acceptable” indicates approval by a regulatory agency of the Federal or a state government or listed in the U.S. Pharmacopeia or other generally recognized pharmacopeia for use in animals, and more particularly in humans.

A “carrier” refers to, for example, a diluent, adjuvant, preservative (e.g., Thimersol, benzyl alcohol), anti-oxidant (e.g., ascorbic acid, sodium metabisulfite), solubilizer (e.g., polysorbate 80), emulsifier, buffer (e.g., Tris HCl, acetate, phosphate), antimicrobial, bulking substance (e.g., lactose, mannitol), excipient, auxiliary agent or vehicle with which an active agent of the present invention is administered. Pharmaceutically acceptable carriers can be sterile liquids, such as water and oils, including those of petroleum, animal, vegetable or synthetic origin. Water or aqueous saline solutions and aqueous dextrose and glycerol solutions are preferably employed as carriers, particularly for injectable solutions. Suitable pharmaceutical carriers are described in “Remington's Pharmaceutical Sciences” by E.W. Martin (Mack Publishing Co., Easton, PA); Gennaro, A. R., Remington: The Science and Practice of Pharmacy, (Lippincott, Williams and Wilkins); Liberman, et al., Eds., Pharmaceutical Dosage Forms, Marcel Decker, New York, N.Y.; and Kibbe, et al., Eds., Handbook of Pharmaceutical Excipients, American Pharmaceutical Association, Washington.

As used herein, the term “subject” refers to an animal, particularly a mammal, particularly a human.

The term “treat” as used herein refers to any type of treatment that imparts a benefit to a patient afflicted with a disease, including improvement in the condition of the patient (e.g., in one or more symptoms), delay in the progression of the condition, etc.

As used herein, the term “prevent” refers to the prophylactic treatment of a subject who is at risk of developing a condition (e.g., aberrant bleeding) resulting in a decrease in the probability that the subject will develop the condition.

A “therapeutically effective amount” of a compound or a pharmaceutical composition refers to an amount effective to prevent, inhibit, treat, and/or lessen the symptoms of a particular disorder or disease.

An “antibody” or “antibody molecule” is any immunoglobulin, including antibodies and fragments thereof, that binds to a specific antigen. As used herein, antibody or antibody molecule contemplates intact immunoglobulin molecules, immunologically active portions of an immunoglobulin molecule (e.g., antigen-binding fragment), and fusions of immunologically active portions of an immunoglobulin molecule.

As used herein, the term “immunologically specific” refers to proteins/polypeptides, particularly antibodies, that bind to one or more epitopes of a protein or compound of interest, but which do not substantially recognize and bind other molecules in a sample containing a mixed population of antigenic biological molecules.

“Linker” refers to a chemical moiety comprising a covalent bond or a chain of atoms that covalently attach at least two compounds. The linker can be linked to any synthetically feasible position of the compounds, but preferably in such a manner as to avoid blocking the compounds desired activity. Linkers are generally known in the art. In a particular embodiment, the linker comprises amino acids. In a particular embodiment, the linker may contain from 0 (i.e., a bond) to about 50 atoms, from 0 to about 10 atoms, or from about 1 to about 5 atoms.

As used herein, the term “small molecule” refers to a substance or compound that has a relatively low molecular weight (e.g., less than 4,000, less than 2,000, particularly less than 1 kDa or 800 Da). Typically, small molecules are organic, but are not proteins, polypeptides, or nucleic acids, though they may be amino acids or dipeptides.

The following examples are provided to illustrate various embodiments of the present invention. The examples are not intended to limit the invention in any way.

### Example 1

To study the effect of CAR-T therapies for AAV NAb eradication, a mouse model was used. Briefly, mice were administered AAV8-FVIII-SQ. Two weeks later, the mice were administered radiation therapy and CAR-T therapy. At days 14, 28, 42, and 56, blood samples were collected and analyzed for the presence of NAb.

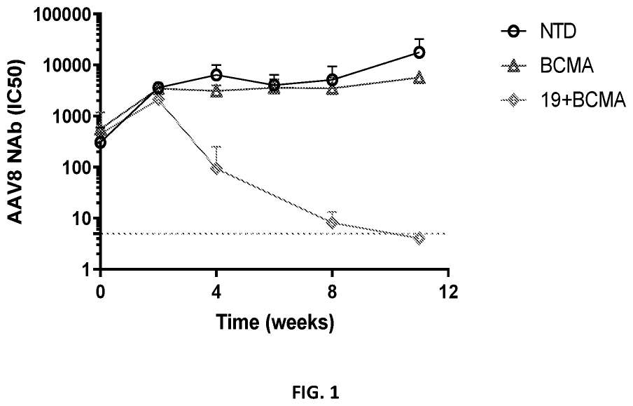

As seen in FIG. 1, the administration of CAR-T against CD19 and BCMA resulted in the dramatic and sustained decrease in the presence of AAV8 NAbs. BCMA CAR-T was insufficient by itself.

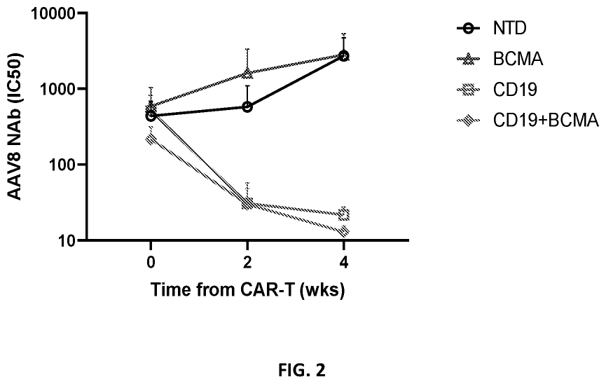

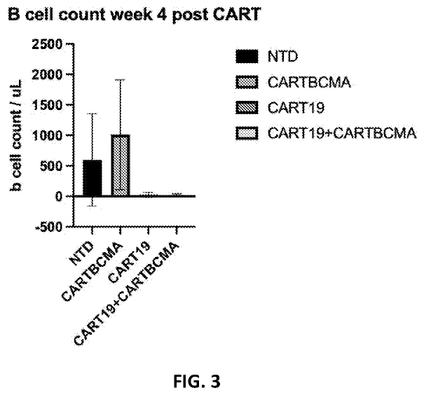

The experiments were repeated with three test groups-BCMA CAR-T, CD19 CAR-T, and CD19 CAR-T with BCMA CAR-T. As seen in FIG. 2, CD19 CAR-T was sufficient to have a sustained decrease in neutralizing antibodies against AAV8. The inclusion of BCMA CAR-T further reduced the amount of neutralizing antibodies present. The B cell count was also determined at 4 weeks post CAR-T. As seen in FIG. 3, CD19 CAR-T and CD19 CAR-T with BCMA CAR-T effectively reduced or eliminated B cells.

### Example 2

Within an estimated 7,000 rare genetic disorders, 95% of patients lack disease modifying therapy. Two thirds of these patients are children. Thus, the burden of genetic disease disproportionally impacts children (Mendell, et al. (2021) Mol. Ther., 29:464-488). Nearly all in vivo gene therapy efforts for inherited disorders currently employ AAV vectors. There are currently ˜150 independent AAV trials, representing a 5-fold increase within 5 years and exponential growth in AAV clinical trials (Kuzmin, et al. (2021) Nat. Rev. Drug Discov., 20:173-174). Consistent with the expansion and success of AAV in the clinic, there are now licensed vectors for spinal muscular atrophy, a form of inherited retinopathy, hemophilia A and B and Duchenne Muscular Dystrophy (DMD) with others anticipated in the short term. Accordingly, AAV is a bona fide novel therapeutic drug class.

The presence of IgG neutralizing antibodies (NAb) limit or preclude the efficacy of systemic AAV vector administration via interfering with cell surface binding and/or causing reticuloendothelial vector clearance preventing target cell transduction (Jiang, et al. (2006) Mol. Ther., 14:452-455; Manno, et al. (2006) Nat. Med., 12:342-347; George, et al. (2017) N. Engl. J. Med., 377:2215-2227; Pipe, et al. (2023) N. Engl. J. Med., 388:706-718; Louis Jeune, et al. (2013) Hum. Gene Ther. Methods 24:59-67; Sun, et al. (2018) Hum. Gene Ther., 29:381-389; Wang, et al. (2017) Gene Ther., 24:49-59). Thus, the presence of NAb has profound therapeutic implications. While lack of assay standardization precludes direct comparisons between studies, multiple groups have universally demonstrated that multi-serotype cross-reactive NAb develop after environmental exposure to wild type AAV (wt-AAV) and/or after AAV vector administration. The seroprevalence of anti-AAV NAb is variable throughout human populations and is dependent on a number of factors including the capsid serotype, geographic region, and age (Boutin, et al. (2010) Hum. Gene Ther., 21:704-712; Li, et al. (2012) Gene Ther., 19:288-294; Kruzik, et al. (2019) Mol. Ther. Methods Clin. Dev., 14:126-133). Generally, NAb against AAV serotype 2 (AAV2) are the most common and can be found in 50% of adults. Anti-AAV 5, 8, and 9 NAb are less prevalent, but can still be found in up to 40% of adults depending on the serotype. AAV serotypes have a high degree of homology both at the peptide sequence and structural level. As such, NAb generated against one serotype can generally cross-neutralize other serotypes. Environmental exposure results in ˜50% prevalence of AAV NAb by age 2 years and persist life-long (Boutin, et al. (2010) Hum. Gene Ther., 21:704-712; Li, et al. (2012) Gene Ther., 19:288-294; Calcedo, et al. (2016) Hum. Gene Ther. Clin. Dev., 27:79-82). Additionally, NAb that develop post AAV vector infusion are typically a log/multiple log-folds higher than NAb resulting from environmental exposure to wt-AAV (Manno, et al. (2006) Nat. Med., 12:342-347; George, et al. (2017) N. Engl. J. Med., 377:2215-2227; George, et al. (2021) N. Engl. J. Med., 385:1961-1973; Long, et al. (2021) Mol. Ther., 29:597-610; Nathwani, et al. (2011) Mol. Ther., 19:876-885; Rangarajan, et al. (2017) N. Engl. J. Med., 377:2519-2530). High-titer, multi-serotype cross reactive NAb that develop post AAV vector infusion persist for up to 15 years (George, et al. (2020) Mol. Ther., 28:2073-2082). Analogous observations have been recapitulated in humans and non-human primates (NHP) by others (Long, et al. (2021) Mol. Ther., 29:597-610; Wang, et al. (2017) Gene Ther., 24:49-59; Aronson, et al. (2019) Hum. Gene Ther., 30:1297-1305).

Overwhelmingly, AAV clinical trials and licensed product labels currently exclude patients with NAb. While NAb titer thresholds vary, the majority of acceptable titers for vector infusion (e.g. <1:5 NAb or <1:50 total antibody [TAb]) are lower than titers observed post environmental AAV exposure and systemic AAV vector infusion. Thus, pre-existing NAb are the most common reason for AAV ineligibility. Additionally, available data suggest unlikely efficacy of repeat vector infusion. This point is relevant in the current state of AAV gene therapy whereby patients will be ineligible for repeat vector administration should therapeutic expression be insufficient, not durable, or if one vector is inferior to another for the same indication.

The only current way to “bypass” NAb is to exclude seropositive patients from receiving vector resulting in an urgent therapeutic need to develop immune modulatory strategies to overcome limitations posed by NAb. Several approaches have been investigated with limited success and minimal human data such as by plasmapheresis (Bertin, et al. (2020) Sci. Rep., 10:864; Chicoine, et al. (2014) Mol. Ther., 22:338-347; Monteilhet, et al. (2011) Mol. Ther., 19:2084-2091), anti-CD20 (rituximab) (Mingozzi, et al. (2013) Gene Ther., 20:417-424); and IgG endopeptidase (imifildase) (Leborgne, et al. (2020) Nat. Med., 26:1096-1101). Despite multiple investigative approaches to reduce or eradicate NAb, none have reliably demonstrated the ability to eradicate or reduce NAb <1:100. While the upper limit threshold NAb titer that permits transduction and therapeutic efficacy is variable based on assay methods, vector dose, capsid, among other factors, the majority of clinical trials exclude subjects with NAb titers>1:5.

Herein, it is shown that rationally designed B cell depletion by targeting surface proteins defining subsets can eradicate NAb to permit initial and repeat therapeutic AAV vector dosing in the presence of NAb.

### Methods

AAV NAb titers were measured via a transduction inhibition assay using a luciferase reporter AAV8 vector. Plasma human factor VIII (hFVIII) antigen levels were measured by ELISA to assess transduction capacity. Flow cytometric assays were performed from peripheral blood to quantify B cells. Animals were euthanized at end of study to quantify B cell subsets in the spleen and bone marrow. Statistical analysis was conducted via GraphPad Prism (v9.0) by mixed effects models or ANOVA with Tukey correction for multiple comparisons.

### Results

Anti-CD19 Containing Therapeutic Regimens Significantly Reduce AAV NAb to ≤1:5, which Meets Criteria for the Majority of Current AAV Clinical Trials or Licensed Vectors

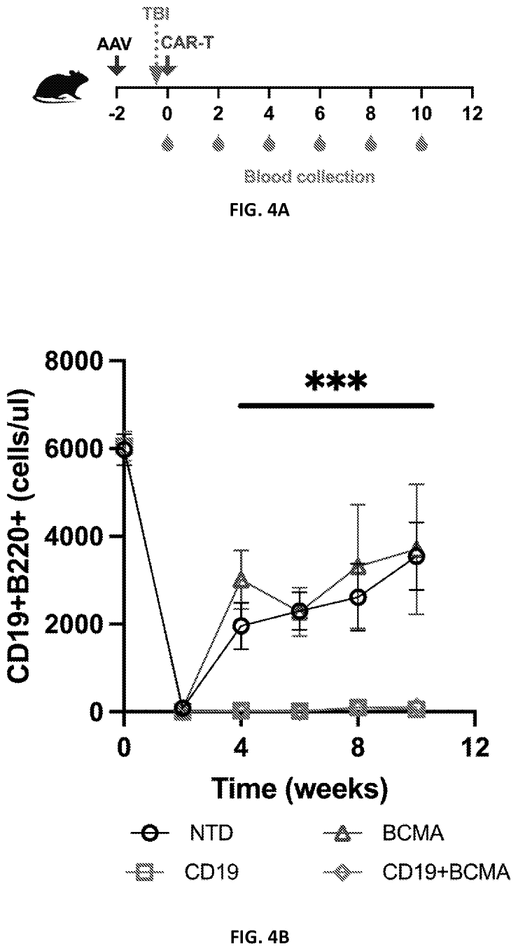

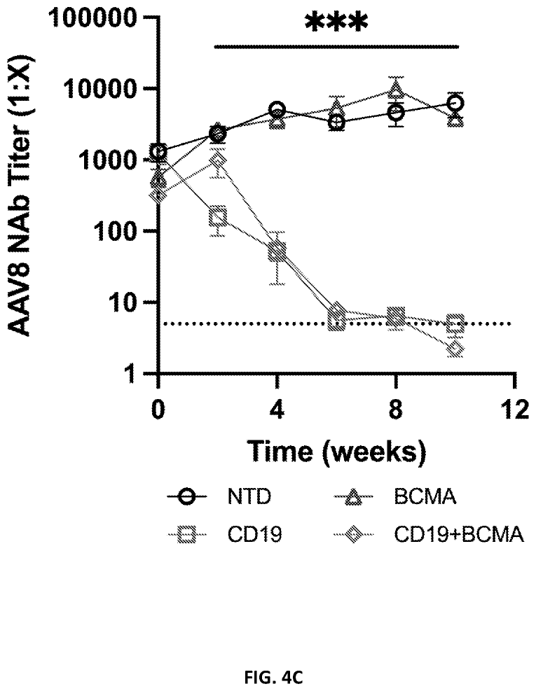

Wild type C57B1/6 mice were immunized with an AAV8 vector (1×10¹¹ vg/mouse) to establish AAV NAb (FIG. 4A). Two weeks later, animals were administered chimeric antigen receptor (CAR) T-cells that were either non-transduced (NTD, controls) or targeted mouse CD19, mouse B-cell maturation antigen (BCMA), or both. Initial AAV exposure resulted in NAb titers prior to CAR-T therapy that were not different between experimental groups (FIG. 4C). Significant and persistent peripheral blood B-cell aplasia was noted starting 2 weeks after CD19 and CD19+BCMA CAR-T infusion (<55.4±96.2 or 115.9.4±90.3 cells/μL, respectively) in contrast to NTD and BCMA (3545.2±2311.3 or 3706.6±2960.9 cells/μL, respectively) CAR-T infusion (p<0.001, mixed model ANOVA) (FIG. 4B). Following CAR-T infusion, NAb titers in the NTD and BCMA-only CAR-T groups continued to rise to greater than 1:3200 whereas mice treated with CD19 or CD19+BCMA CAR-T regimens had mean titers of 1:5 (p<0.01) and 1:2 (p<0.05) at week 10, respectively (FIG. 4C). There was no significant difference between CD19 alone or CD19+BCMA CAR-T regimens (although the inclusion of BCMA did slightly lower NAb titer by week 10) and the NTD or BCMA CAR-T regimens, thus these groups were combined for subsequent re-challenge analysis.

Mice with Pre-existing High Titer AAV NAb Were Treated with Anti-CD19 Containing CAR-T Regimens to Reduce AAV NAb to ≤1:5. Thereafter, Mice Were Re-challenged with AAV Vector to Demonstrate Target Tissue Transduction and Transgene Expression.

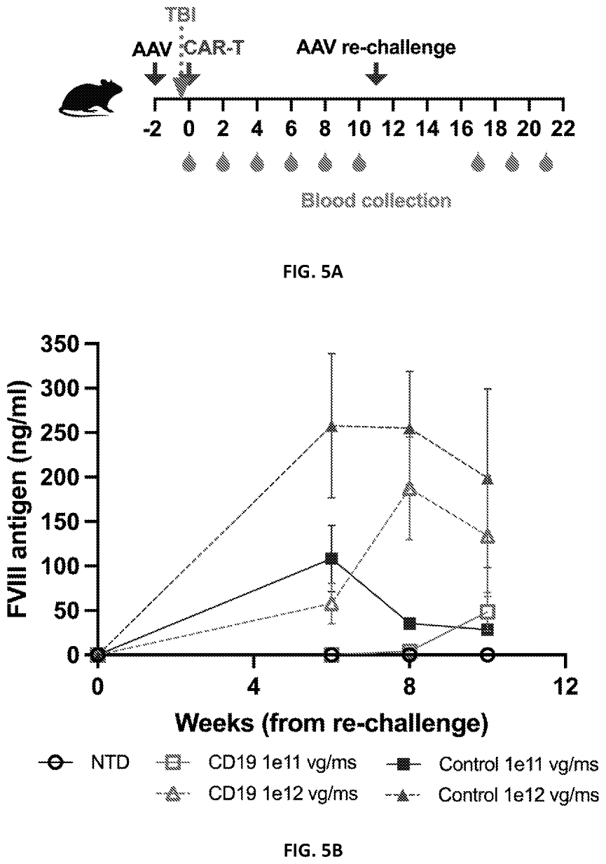

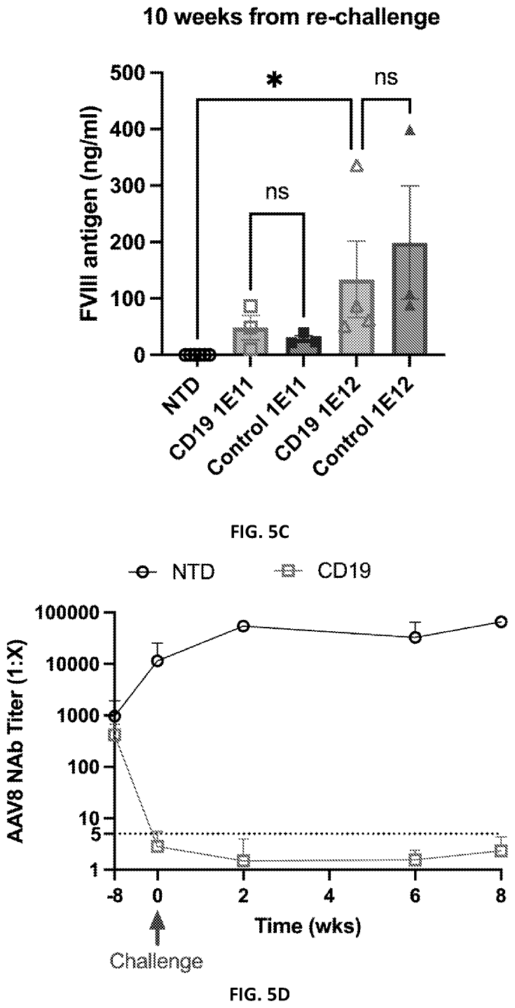

Eleven weeks after initial CAR-T therapy and 13 weeks from initial AAV injection, mice were rechallenged with an AAV8 vector carrying human FVIII at doses of 1×10¹¹ or 1×10¹² vg/mouse (FIG. 5A). In parallel, control animals that were not previously immunized with AAV but did receive CD19 CAR-T were infused with AAV8-hFVIII vector at 1×10¹¹ and 1×10¹² vg/ms. In previously immunized mice, upon AAV8-FVIII re-challenge, FVIII antigen levels inversely correlated with NAb titer (Spearman r=−0.56, p<0.05). In these mice, there was no FVIII expression in the NTD group whereas FVIII antigen was detected in the CD19 CAR treated mice (FIG. 5B). Previously immunized CD19 CAR-T mice rechallenged with AAV at 1×10¹¹ vg/ms had mean FVIII expression of 48.4±38.2 ng/ml, comparable to control non-immunized mice who had FVIII antigen levels of 28.4±9.6 ng/ml (p>0.05, FIG. 5C). Similarly, previously AAV immunized CD19 CAR-T treated mice given 1×10¹² vg/ms AAV8-hFVIII expressed 133.7±135.7 ng/ml compared to 198.9±173.9 ng/ml in unimmunized mice (p>0.05, FIG. 5C). CAR-19 treated mice did not develop high titer AAV following AAV vector rechallenge, demonstrating CAR-19 prevents high titer AAV NAb induction post AAV (FIG. 5D).

CD19-containing CAR-T Regimens Effectively Deplete B cells in the Bone Marrow and Spleen

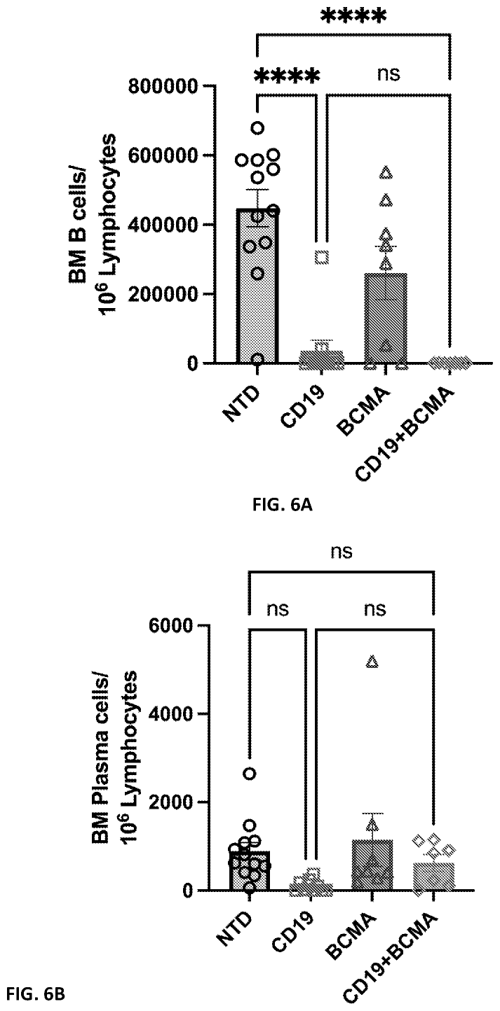

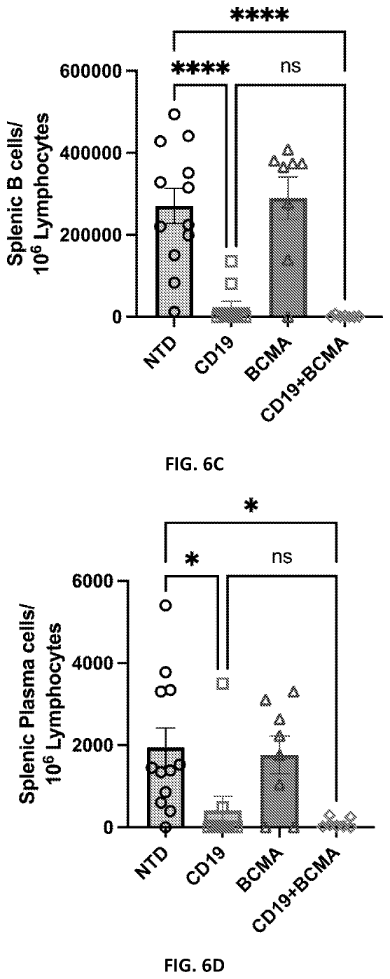

All mice were euthanized at 24-28 weeks following CAR-T therapy. Spleen and bone marrow was harvested for mononuclear cell isolation and flow cytometric quantification of B cells (CD19+B220+) and plasma cells (CD138+TACI+). In bone marrow, CD19-containing CAR-T regimens significantly depleted total B cells (FIG. 6A) compared to NTD or BCMA CAR-T regimens (p<0.001). Compared to NTD (886.7±674.0 cells/million lymphocytes) and BCMA (1145±1685) groups, CD19 CAR-T (102±126) trended towards decrease in bone marrow plasma cells (FIG. 6B). In the spleen, CD19-containing CAR-T regimens effectively ablated total B cells (p<0.001, FIG. 6C) and plasma cells (p<0.001, FIG. 6D) compared to the NTD and BCMA groups. These data indicate that effective B cell depletion in lymphoid organs in addition to peripheral blood can deplete AAV NAb.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

1. A method for reducing neutralizing antibodies in a subject in need thereof, said method comprising administering a CD19 targeting agent, a B-cell activating factor (BAFF) targeting agent, a B-cell maturation antigen (BCMA) targeting agent, and/or a transmembrane activator and CAML interactor (TACI) targeting agent to a subject.

2. The method of claim 1, wherein said method comprises administering a CD19 targeting agent and, optionally, a BCMA targeting agent.

3. (canceled)

4. The method of claim 1, wherein the neutralizing antibodies are anti-adeno-associated virus (AAV) neutralizing antibodies.

5. The method of claim 1, wherein the targeting agent is selected from the group consisting of antibodies, antigen binding antibody fragments, immunotoxins, antibody drug conjugates, small molecules, cytotoxic agents, and chimeric antigen receptor T cells (CAR-T).

6. The method of claim 1, wherein the targeting agent comprises a monoclonal antibody.

7. (canceled)

8. The method of claim 1, wherein the targeting agent reduces or depletes B cells in said subject.

9. The method of claim 1, wherein the neutralizing antibody titer in the subject is reduced to <1:10.

10. A method for reducing or depleting B cells in a subject in need thereof, said method comprising administering a CD19 targeting agent, a B-cell activating factor (BAFF) targeting agent, a B-cell maturation antigen (BCMA) targeting agent, and/or a transmembrane activator and CAML interactor (TACI) targeting agent to a subject.

11. The method of claim 10, wherein said method comprises administering a CD19 targeting agent and, optionally, a BCMA targeting agent.

12. (canceled)

13. The method of claim 10, wherein the B cells are plasma cells.

14. The method of claim 10, wherein the targeting agent is selected from the group consisting of antibodies, antigen binding antibody fragments, immunotoxins, antibody drug conjugates, small molecules, cytotoxic agents, and chimeric antigen receptor T cells (CAR-T).

15. The method of claim 10, wherein the targeting agent comprises a monoclonal antibody.

16. (canceled)

17. A method for administering a viral vector to a subject, said method comprising:
i) administering a CD19 targeting agent, a B-cell activating factor (BAFF) targeting agent, a B-cell maturation antigen (BCMA) targeting agent, and/or a transmembrane activator and CAML interactor (TACI) targeting agent to the subject; and
ii) administering the viral vector to the subject.

18. The method of claim 17, wherein said viral vector is an adeno-associated virus (AAV) vector, optionally wherein said AAV vector is for AAV gene therapy.

19. (canceled)

20. The method of claim 17, wherein step i) occurs before step ii).

21. The method of claim 17, wherein said subject has neutralizing antibodies against the viral vector prior to treatment.

22. The method of claim 17, wherein said method comprises administering a CD19 targeting agent and, optionally, a BCMA targeting agent.

23. (canceled)

24. The method of claim 21, wherein the neutralizing antibodies are anti-adeno-associated virus (AAV) neutralizing antibodies.

25. The method of claim 21, wherein the neutralizing antibodies are alloantibodies.

26. The method of claim 17, wherein the targeting agents are selected from the group consisting of antibodies, antigen binding antibody fragments, immunotoxins, antibody drug conjugates, small molecules, cytotoxic agents, and chimeric antigen receptor T cells (CAR-T).

27. The method of claim 17, wherein the targeting agent comprises a monoclonal antibody.

28. (canceled)
